(19) United States
(12) Patent Application Publication
(10) Pub. No.: US 2015/0038785 A1
(43) Pub. Date: Feb. 5, 2015

(54) INTEGRATED ENDOSCOPE IRRIGATION

(71) Applicant: MEDIGUS LTD., Omer (IL)

(72) Inventors: Amir Govrin, Ramat Gan (IL); Yekaterina Dlugach, Beer Sheva (IL); Tsafrir Kolatt, Zichron Ya'akov (IL)

(73) Assignee: MEDIGUS Ltd, Omer (IL)

(21) Appl. No.: 14/385,185

(22) PCT Filed: Feb. 28, 2013
(86) PCT No.: PCT/IL2013/050170

§ 371 Date: Sep. 15, 2014

Related U.S. Application Data
(60) Provisional application No. 61/616,097, filed on Mar. 27, 2012.

(51) Int. Cl. A61B 1/015; A61B 1/005; A61B 1/00

(52) U.S. Cl. CPC A61B 1/015; A61B 1/00071; A61B 1/0008; A61B 1/0057; A61B 1/00091
USPC 600/108; 600/156; 600/109; 600/129; 600/104

(57) ABSTRACT

In a first aspect the invention is an endoscopic device in which the empty spaces between the plurality of tubes, wires, and cables are utilized as a channel that enables liquid or gas to flow from the handle section to the distal tip. In a second aspect the invention is a method of reducing the diameter of an endoscope device by utilizing the empty spaces between the plurality of tubes, wires, and cables as a channel that enables liquid or gas to flow from the handle section to the distal tip. In a third aspect the invention is a distal tip comprising a pattern of alternating grooves and lands on its outer surface and a cap that fits tightly over the lands. The tight fitting cap changes the grooves into closed channels through which liquid or gas can flow.

### FIELD OF THE INVENTION

The invention is from the field of medical devices. More specifically, the invention is from the field of small diameter endoscopic devices.

### BACKGROUND OF THE INVENTION

In various medical applications there are many advantages for using small diameter endoscopes and laparoscopes (collectively called endoscopes or endoscopic devices herein) having, for example, a maximum outer diameter of 3.2 mm. Most importantly small diameter endoscopes can be introduced to desired locations within the body through small diameter natural orifices and lumens. Also in cases where introduction of the endoscope may be irritating, a small diameter endoscope may mitigate such phenomena. An example of a procedure in which small diameter endoscopes can be useful is transnasal endoscopy that in some cases may replace trans-oral endoscopy. Moreover, small diameter endoscopes may be introduced into body cavities by single incision laparoscopy, wherein the incision itself is of minimal dimensions.

By its nature, endoscopy entails incorporating many components adapted to perform various functions within a single elongated instrument. This fact sometimes conflicts with the desire for minimum diameter and size in general. Among these components are: vision mechanisms, e.g. video cameras; illumination means, e.g. optical fibers or LEDS; articulation means; tissue collection elements or other surgical tools; irrigation, insufflation, and more.

One of the ways to accommodate as many components and functions as possible to decrease the size of each individual component, e.g., using a smaller size camera or a smaller size fiber bundle. However, this is not always possible, there are limits to how much reduction can be achieved, and each size reduction has its cost in terms of performance and assembly complexity.

It is therefore a purpose of the present invention to provide a method of reducing the diameter of endoscope devices.

It is another purpose of the present invention to provide a method of providing an endoscopic device with more components without increasing the cross section of the insertion tube.

Further purposes and advantages of this invention will appear as the description proceeds.

### SUMMARY OF THE INVENTION

In a first aspect the invention is an endoscopic device that comprises a handle section, an insertion tube connected to the handle section, a distal tip at the distal end of the insertion section, and a plurality of tubes, wires, and cables that pass through the interior of the insertion tube. The endoscopic device of the invention is characterized in that the empty spaces between the plurality of tubes, wires, and cables are utilized as a channel that enables liquid or gas to flow from the handle section to the distal tip.

In embodiments of the invention the endoscopic device comprises at least one of:
(a) An articulation section located at the distal end of the insertion tube proximally to the distal tip. The articulation section is activated by cables or wires that pass through tubes that extend the length of the interior of the insertion tube from the handle section to the articulation section.
(b) An imaging device located in the distal tip. The imaging device is activated by power delivered to it and transmits images gathered by it via one or more cables, wires, or optical fibers that pass through one or more tubes that extend the length of the interior of the insertion tube from the handle section to the distal tip.
(c) Illumination means located in the distal tip. The illumination means are activated by wires or optical fibers that pass through one or more tubes that extend the length of the interior of the insertion tube from the handle section to the distal tip.
(d) One or more working channels that pass through the interior of the insertion tube from the handle section to the distal tip.
(e) One or more other components each of which is located at a location on the insertion tube or on the distal tip and is associated with a tube, wires, or cable that passes through the interior of the insertion tube from the handle section to the location.

In embodiments of the endoscopic device of the invention the handle section comprises components of an articulation mechanism including articulation cylinders that are sealed by gaskets, which are adapted to enable movement of the cables or wires that pass through the insertion tube to steer the articulation section without leakage of liquid or gas between the handle section and the insertion tube.

In embodiments of the endoscopic device of the invention the imaging device is a video camera.

In embodiments of the endoscopic device of the invention the components located at a location on the insertion tube or on the distal tip are selected from: lasers and radio frequency generators.

Embodiments of the endoscopic device of the invention comprise at least one nozzle on the distal tip. In these embodiments the liquid or gas that flows from the handle to the distal tip through the empty spaces between the plurality of tubes, wires, and cables that pass through the interior of the insertion tube flows through the at least one nozzle.

In embodiments of the endoscopic device of the invention the distal tip comprises a pattern of alternating grooves and lands on its outer surface and a cap that fits tightly over the lands. The tight fitting cap changes the grooves into closed channels through which liquid or gas flowing through the empty spaces between the plurality of tubes, wires, and cables in the insertion tube can continue on its way to the at least one nozzle.

In a second aspect the invention is a method of reducing the diameter of an endoscope device that comprises a handle section, an insertion tube connected to the handle section, a distal tip at the distal end of the insertion section, and a plurality of tubes, wires, and cables that pass through the interior of the insertion tube. The method od the invention comprises utilizing the empty spaces between the plurality of tubes, wires, and cables as a channel that enables liquid or gas to flow from the handle section to the distal tip.

In embodiments of the method of the invention the endoscopic device comprises at least one nozzle on the distal tip and the liquid or gas that flows through the empty spaces between the plurality of tubes, wires, and cables that pass through the interior of the insertion tube from the handle section of the endoscope to the at least one nozzle is used for at least one of the following purposes: irrigation, insufflation, suction, cooling, heating, staining tissue, and therapy.

In a third aspect the invention is a distal tip comprising a pattern of alternating grooves and lands on its outer surface and a cap that fits tightly over the lands. The tight fitting cap changes the grooves into closed channels through which liquid or gas can flow.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1 schematically shows some of the interior components of the handle section of an endoscopic device;

FIG. 2 schematically shows the articulation section and distal tip at the proximal end of an insertion tube that is connected to the handle section shown in FIG. 1;

FIG. 3 schematically shows an enlarged view of the distal tip shown in FIG. 2;

FIG. 4 schematically shows the face of the distal tip shown in FIG. 2; and

FIG. 5 schematically shows a cross-section of a typical insertion tube at a location between the handle and the articulation section.

### DETAILED DESCRIPTION OF EMBODIMENTS OF THE INVENTION

Endoscopic devices are comprised of a handle section, an elongated insertion tube, and a distal tip at the end of the insertion section. An articulation section is often included at the distal end of the insertion section just before the distal tip to allow the distal tip to be deflected and to aid in steering the insertion tube through bodily lumens to the location where the observations or procedure are to be carried out. To prevent bodily fluids, tissue, or debris from entering the insertion tube, it and the articulation section are encased in a sheath of polymeric material.

The present invention is directed to endoscopic devices that typically comprise at least an imaging device, e.g. a video camera and accompanying illumination means in the distal tip and an articulation section that is activated by cables or wires that pass through the interior of the insertion tube from an articulation steering mechanism located in the handle section. Endoscopic devices frequently also comprise one or more working channels, through which surgical tools, e.g. forceps, and therapy devices, e.g. lasers or RF generators, can be introduced from the handle section to the space beyond the distal tip in order to collect samples or carry out various procedures. In addition there are frequently channels for other purposes, e.g. irrigation water or air to clean the camera lens, gas for insufflation, dye for staining tissue, liquid for cooling (or heating), and gas or liquid for therapy, e.g. delivery of drugs or medicine.

In present day endoscopic devices each of the working channels and the channels for irrigation and insufflation are small tubes that run through the insertion tube from handle to distal tip. As an alternative to having separate tubes for each component or function, endoscopes may comprise a multi-lumen tube which contains separate lumens for each component. Also the optical fibers or electric wires for the illumination means and the power and signal wires to and from the camera pass through similar tubes. All of these individual tubes and the articulation cables or wires are tightly packed into the interior of the insertion tube.

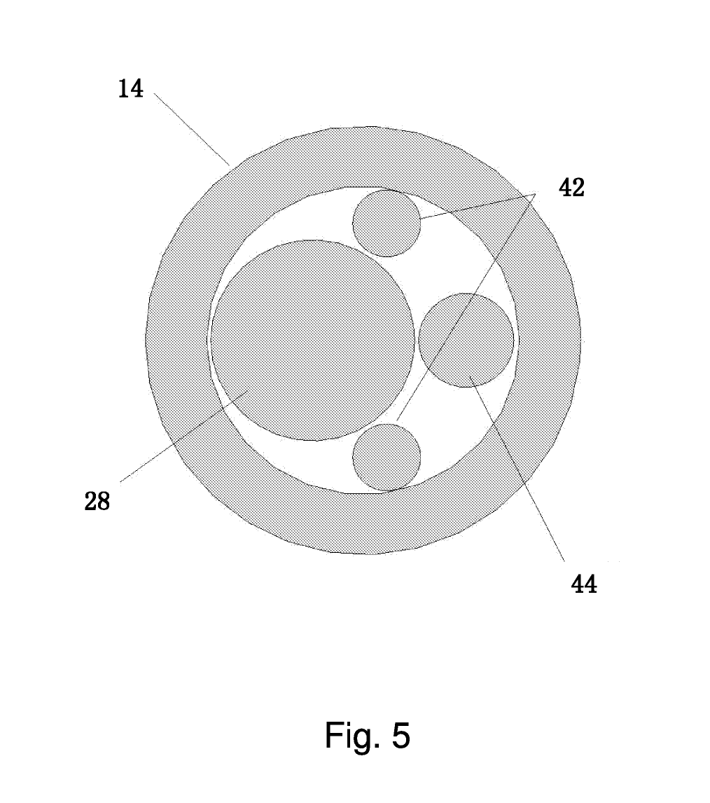

FIG. 5 schematically shows a cross-section of a typical insertion tube at a location between the handle and the articulation section. In the figure can be seen how the illumination fibers 28, camera cable 44, and articulation cables 42 are fit into the interior of insertion section 14. In this example, the minimum inner diameter of insertion tube 14 is determined by the diameters of the illumination fibers 28 and camera cable 44.

As can be seen from FIG. 5, packing a plurality of tubes having a circular cross-section into a larger cylindrical tube inevitable means that there will be empty spaces between the tubes that are not utilized. The current invention makes use of these spaces to allow fluid to flow from the handle of the endoscope to the distal tip. In this way it is not necessary to have a separate irrigation/insufflation/cooling/dye/therapy channel and as a result the overall diameter of the insertion tube can be reduced.

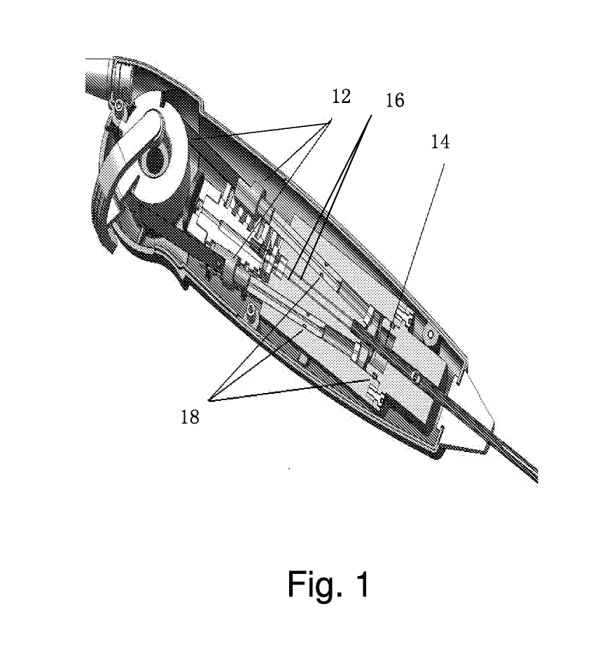

FIG. 1 to FIG. 4 illustrate one embodiment of an endoscope built according to the present invention.

FIG. 1 schematically shows the handle section of an embodiment of an endoscopic device with the cover partially removed to reveal some of the interior components. Shown in FIG. 1 are the articulation handle, articulation drum, and articulation cylinders of articulation mechanism 12; insertion tube 14; illumination fibers and power and signal wires 16; and gaskets 18.

When the cover of the handle section is in place it presses against the gaskets 18 forming an air and water tight compartment in the volume defined by the gaskets. In particular the two small gaskets seal the articulation cylinders and enable movement of the steering cables or wires without leakage of fluid. An inlet port (not shown in the figure) allows water for irrigation or gas for insufflation to be introduced into this compartment.

As will be described herein below, the water or gas flows out of the compartment in the handle into and through the insertion tube and exits the endoscope through a nozzle (or a set of nozzles) located on the distal tip.

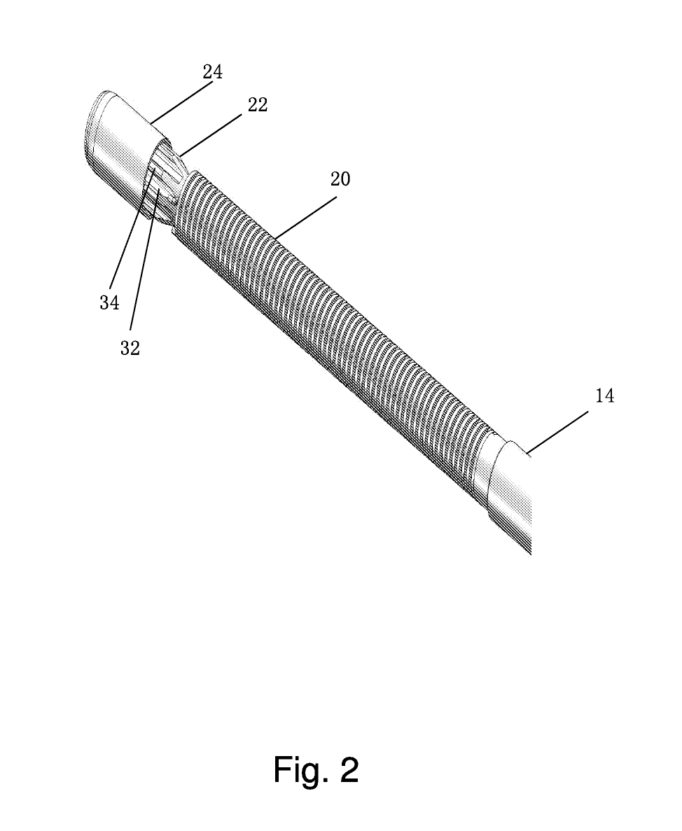

FIG. 2 schematically shows the distal end of an insertion tube 14 that is connected at its proximal end to the handle section shown in FIG. 1. The sheath that covers the insertion tube has been removed to reveal articulation section 20 and distal tip 22. In contrast to conventional distal tips, which have smooth external surfaces that are shaped to minimize trauma as the endoscope is advanced to the site of the procedure, a pattern of alternating grooves 32 and lands 34 is created on the outer surface of distal tip 22. A cap 24, which fits tightly over the lands 34 provides the smooth outer surface. The tops of lands 34 are pressed against the inside wall of cap 24 forming hermetic seals that converts grooves 32 into closed channels through which water or gas flowing through the insertion tube can continue on its way to a nozzle on the distal end of the endoscope.

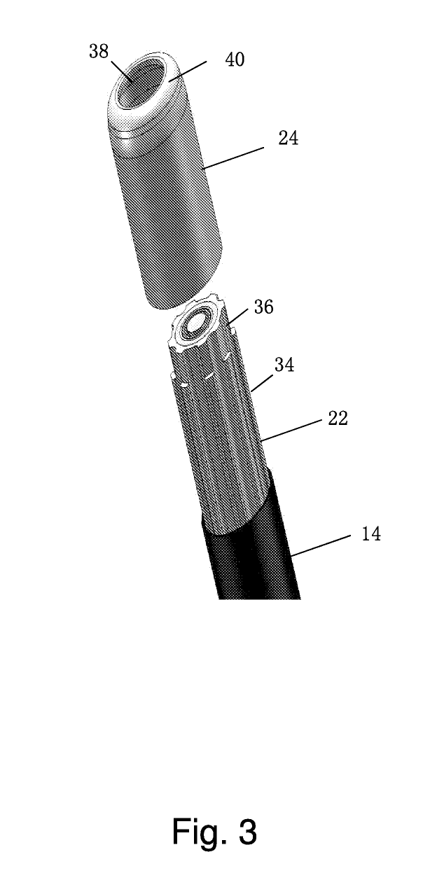

As shown in FIG. 3, a circular portion at the center of the distal surface of cylindrical cap 24 is removed leaving a hole 38 surrounded by an annular curved portion 40. As can also be seen in the same figure, a part 36 of the distal end of each land 34 is removed to form an annular space around the distal tip under curved portion 40 of cap 24.

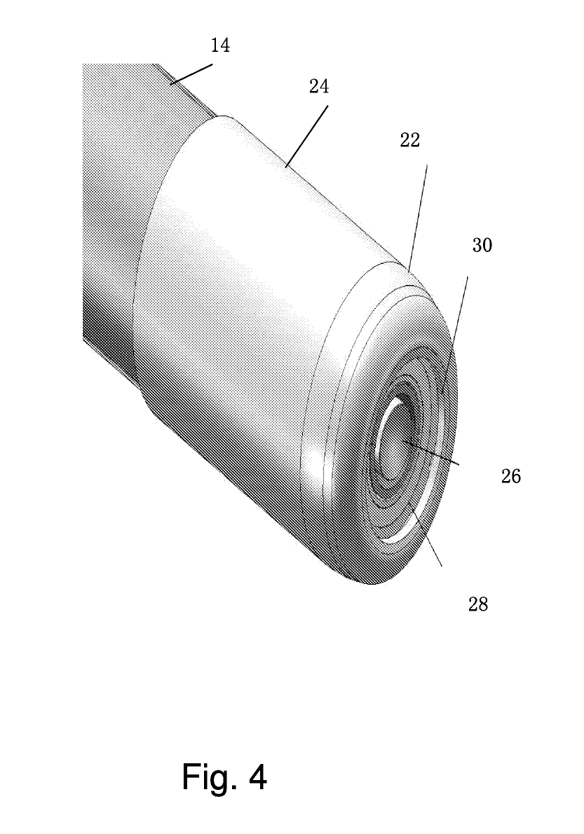

With the insertion section 14, including the articulation section, completely covered with the sheath and the cap 24 in place over the distal tip 22 as shown in FIG. 4, the inside of the endoscope is hermetically isolated from the outside with the exception of a small gap around the circumference of hole 38 in cap 24. The shape of curved surface 40 causes the gap to function as a circular nozzle 30. With the configuration described herein, the video camera 26 is located at the center of the distal tip surrounded by illumination fibers 28. Water or gas introduced into the interior of the handle enters the insertion tube 14 and flows through the empty spaces between the tubes and cables that pass through insertion tube 14 and articulation section 20. On reaching distal tip 22, the water or gas flows through grooves 32 and out of the distal end through circular nozzle 30. The components of the distal tip are configured such that water or gas exiting through circular nozzle 30 is sprayed over the illumination fibers 28 and objective lens of camera 26 keeping them clean.

It is noted that only a very basic embodiment of an endoscopic device has been described herein in order to illustrate the principle of the invention. In addition to a camera and illumination means, the endoscope may comprise one or more working channels, and other components located on the distal tip, e.g. ultrasound transducers. All of these require their own tube that must be integrated into the interior of the insertion tube. In these embodiments, eliminating the need for a separate irrigation and/or insufflation channel by utilizing the spaces between the other tubes for the passage of water or gas as taught by the present invention will play an important role in reducing the overall diameter of the insertion tube.

In other embodiments the grooves 32 can be configured to direct the liquid or gas to one or more of any type of nozzle that is used in the art.

Although embodiments of the invention have been described by way of illustration, it will be understood that the invention may be carried out with many variations, modifications, and adaptations, without exceeding the scope of the claims.

1. An endoscopic device that comprises a handle section, an insertion tube connected to said handle section, a distal tip at the distal end of said insertion section, and a plurality of tubes, wires, and cables that pass through the interior of said insertion tube;
wherein, the empty spaces between said plurality of tubes, wires, and cables are utilized as a channel that enables liquid or gas to flow from said handle section to said distal tip.

2. The endoscopic device of claim 1 comprising at least one of:
(a) an articulation section located at the distal end of the insertion tube proximally to the distal tip, said articulation section activated by cables or wires that pass through tubes that extend the length of the interior of said insertion tube from the handle section to said articulation section;
(b) an imaging device located in said distal tip, said imaging device activated by power delivered to it and transmitting images gathered by it via one or more cables, wires, or optical fibers that pass through one or more tubes that extend the length of the interior of said insertion tube from said handle section to said distal tip;
(c) illumination means located in said distal tip, said illumination means activated by wires or optical fibers that pass through one or more tubes that extend the length of the interior of said insertion tube from said handle section to said distal tip;
(d) one or more working channels that pass through the interior of said insertion tube from said handle section to said distal tip;
(e) one or more other components each of which is located at a location on said insertion tube or on said distal tip and is associated with a tube, wires, or cable that passes through the interior of said insertion tube from said handle section to said location.

3. The endoscopic device claim 2, wherein the handle section comprises components of an articulation mechanism including articulation cylinders that are sealed by gaskets, which are adapted to enable movement of the cables or wires that pass through the insertion tube to steer the articulation section without leakage of liquid or gas between said handle section and said insertion tube.

4. The endoscopic device claim 2, wherein the imaging device is a video camera.

5. The endoscopic device of claim 2, wherein the components located at a location on the insertion tube or on the distal tip are selected from: lasers and radio frequency generators.

6. The endoscopic device of claim 1, comprising at least one nozzle on the distal tip;
wherein, the liquid or gas that flows from the handle to said distal tip through the empty spaces between the plurality of tubes, wires, and cables that pass through the interior of the insertion tube flows through said at least one nozzle.

7. The endoscopic device of claim 6, wherein, the distal tip comprises a pattern of alternating grooves and lands on its outer surface and a cap that fits tightly over said lands; thereby changing said grooves into closed channels through which liquid or gas flowing through the empty spaces between the plurality of tubes, wires, and cables in the insertion tube can continue on its way to the at least one nozzle.

8. A method of reducing the diameter of an endoscope device that comprises a handle section, an insertion tube connected to said handle section, a distal tip at the distal end of said insertion section, and a plurality of tubes, wires, and cables that pass through the interior of said insertion tube;
said method comprising utilizing the empty spaces between said plurality of tubes, wires, and cables as a channel that enables liquid or gas to flow from said handle section to said distal tip.

9. The method of claim 8, wherein the endoscopic device comprises at least one nozzle on the distal tip and the liquid or gas that flows through the empty spaces between the plurality of tubes, wires, and cables that pass through the interior of the insertion tube from the handle section of said endoscope to said at least one nozzle is used for at least one of the following purposes: irrigation, insufflation, suction, cooling, heating, staining tissue, and therapy.

10. A distal tip comprising a pattern of alternating grooves and lands on its outer surface and a cap that fits tightly over said lands; thereby changing said grooves into closed channels through which liquid or gas can flow.
